SEQUENCE LISTING (7 sequences)

SEQ ID NO: 1 - protein, 114 aa, Artificial Sequence
  E4 orf4-encoded protein
MVLPSLPSPLLLETQSSCIAWLGFAYATVDDFLRTIRCDGVLITTEASILLTNLRVWLYLNYHTEHAKRQDRRRRSVCHARTWFCFRKYDYVRRSIWHDTTTNTISVVSAHSVQ

SEQ ID NO: 2 - protein, 114 aa, Artificial Sequence
  E4 orf4-encoded protein
MILPSLPSPLLLETQSSCIAWLGFAYATVDDFLRTIKYDEVLITTEASILLTNLRVWLYLNYQTEHTKRQDRRRRSVCHARTWFCFRKYDYVRRSIWHDTTTNTISVVSAHSVQ

SEQ ID NO: 3 - protein, 114 aa, Artificial Sequence
  E4 orf4-encoded protein
MILPSLPSPLLLETQSSCIAWLGFAYATVDDFLRTIKYDGVLITTEASILLTNLRVWLYLNYQTEHTKRQDRRRRSVCHARTWFCFRKYDYVRRSIWHDTTTNTISVVSAHSVQ

SEQ ID NO: 4 - DNA, 37021 bp, Artificial Sequence
  Ad5 including E4 orf4-encoded protein
  LTR 1..103: /note=“L-ITR”
  promoter 380..625: /note=“CMVs promoter”
  gene 727..1646: /gene=“E1A gene, codons for amino acids 4 to 25 of wild-type deleted”
  misc_feature 837..857: /note=“RNAi target site”
  misc_feature 886..909: /note=“RNAi target site”
  misc_feature 1684..1764: /note=“heterologous target-sites for RNAi inserted into the 3?UTR of E1A, equivalent to E1A-886..909, E1A-837..857, Prot IX?7353..7374)”
  promoter 1813..1910: /note=“promoter for E1B”
  gene 1911..2441: /gene=“E1B-19k”
  gene 2216..3706: /gene=“E1B-55k”
  protein_bind 3731..4320: /note=“IRES”
  CDS 4349..5053: /note=“FLT3L” /transl_table=1
  misc_feature 5054..5116: /note=“2A”
  gene 5117..5392: /gene=“MIP1A/CCL3”
  misc_feature 5393..5455: /note=“2A”
  gene 5456..5800: /gene=“XCL1”
  promoter 5872..6230: /note=“prMinRGC”
  misc_feature 6259..6321: /note=“inhibitory shRNA for E1A (1)”
  misc_feature 6259..7064: /note=“shRNA network”
  misc_feature 6387..6444: /note=“inhibitory shRNA for E1A (2)”
  misc_feature 6511..6567: /note=“inhibitory shRNA for Portein IX”
  misc_feature 6634..6690: /note=“inhibitory shRNA for E4 orf6 (1)”
  misc_feature 6756..6816: /note=“inhibitory shRNA for E4 orf6 (2)”
  misc_feature 6882..6939: /note=“inhibitory shRNA for TP”
  misc_feature 7006..7064: /note=“inhibitory shRNA for AdPol”
  gene 7332..7754: /note=“Protein IX, part of backbone”
  gene 8920..17842: /note=“polymerase, Ad-Pol, part of backbone”
  gene 12306..17842: /gene=“terminal protein, TP, part of backbone”
  gene 17878..19593: /gene=“Protein III Penton, part of backbone”
  gene 22563..25421: /gene=“hexon, part of backbone”
  gene 26164..27753: /gene=“reverse complementary sequence of DNA-binding protein, dbd, part of backbone”
  gene 27782..30205: /gene=“100k, part of backbone”
  misc_feature 31589: /note=“deltaE3, (equivalent to del. of nt 28137 ? 30818 in the wildtype virus)”
  gene 32108..33853: /gene=“Fiber, part of backbone”
  gene 33980..35143: /gene=“reverse complementary sequence of E4 orf6/7”
  gene 35064..35408: /gene=“reverse complementary sequence of hybrid E4 orf4 Ad5/Ad11, amino acid sequence is SEQ ID NO 1”
  misc_feature 35419..35770: /note=“reverse complementary sequence of inactivated E4 orf3 locus”
  terminator 35747..35749: /note=“stop codon E4orf3”
  gene 35782..36175: /gene=“reverse complementary sequence of E4orf2”
  gene 36207..36593: /gene=“reverse complementary sequence of E4orf1”
  LTR 36919..37021: /note=“R-ITR”
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatctctagcatcgtaactataacggtcctaaggtagcgaaagctagtaccatggtgatgcggttttggcagtacatcaatggcggtggatagcggtccgactcacggggatttccaagtctccaccccattgacgtcaatgggagtttgttttggcaccaaaatcaacgggactttccaaaatgtcgtaacaactccgccccacggacgcaaatgggccgtaggcgtgtacggtgggcggtctatataagcccggctggtttagtgaaccgtcagatcctcgagggcgtgtagtgtatttatacccggtgagttcctcaagaggccactcttgagtgccagcgagtagagttttctcctccgagccgctccgacaccgggactgaaaatgagacatgaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacacacaggaggcggtttcgcagatttttacacacagaagggattgacttactcacttttacacacaggaagcattgtgagctcatatgagacccaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgatcacgcgtttcgaacgatttaaatttaaatatgacagtgctggcgccagcctggagcccaacaacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggctgctgcctggtgcctgcactggcagaggacgcggcggaggacaccccgccctggggagcaggtgccccccgtccccagtccccaggacctgctgcttgtggagcactccggagccacgaacttctctctgttaaagcaagcaggagacgtggaagaaaaccccggtcccatgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctggagctgagtgcctccggagccacgaacttctctctgttaaagcaagcaggagacgtggaagaaaaccccggtcccatgagacttctcatcctggccctccttggcatctgctctctcactgcatacattgtggaaggtgtagggagtgaagtctcagataagaggacctgtgtgagcctcactacccagcgactgccagttagcagaatcaagacctacaccatcacggaaggctccttgagagcagtaatttttattaccaaacgtggcctaaaagtctgtgctgatccacaagccacatgggtgagagacgtggtcaggagcatggacaggaaatccaacaccagaaataacatgatccagaccaagccaacaggaacccagcaatcgaccaatacagctgtgactctgactggctagctcgagaataaaggtaccttattttcattagatttgtgtgttggttttttgtgtggcggcctcgagatgggagagcttgatgccaggcaagtccaggcaggccaggcaagtccaggcaggccaggcaagtccaggcaggccaggcaagtccaggcaggccaggcaagtccaggcaggccaggcaagtccaggcaggccaggcaagtccaggcaggccaggcaagtccaggcaggccaggcaagtccaggcaggccaggcaagtccaggcagggccaggcaagtccaggcaggccaggcaagtccaggcaggccaggcaagtccaggcaggatcgaattcgagctcgtacggtgggagggctatataagcagacctcgtttagtgaaccgtcagatcgcctggagacgaagcttgctagcggatcctcgagaaggtatattgctgttgacagtgagcgagaagggattgacttactcacttttgtgaagccacagatgaaaagtgagtaagtcaatcccttcgtgcctactgcctcggaattcactagcggatcctcgagaaggtatattgctgttgacagtgagcgaggaggcggtttcgcagattttgtgaagccacagatgaaaatctgcgaaaccgcctccgtgcctactgcctcggaattcactagcggatcctcgagaaggtatattgctgttgacagtgagcgaggaagcattgtgagctcatatgtgaagccacagatgatatgagctcacaatgcttccgtgcctactgcctcggaattcactagcggatcctcgagaaggtatattgctgttgacagtgagcgagacaggaaaccgtgtggaatagtgaagccacagatgtattccacacggtttcctgtcgtgcctactgcctcggaattcactagcggatcctcgaaaggtatattgctgttgacagtgagcgaccatgtttaatcagaggtttatagtgaagccacagatgtataaacctctgattaaacatgggtgcctactgcctcggaattcactagcggatcctcgagaaggtatattgctgttgacagtgagcgacggagtacctacacaacaattgtgaagccacagatgaattgttgtgtaggtactccggtgcctactgcctcggaattcactagcggatcctcgagaaggtatattgctgttgacagtgagcgagcgaatacgtgcagctaaacatgtgaagccacagatgatgtttagctgcacgtattcgcgtgcctactgcctcggaattcactagctagtcatatgctgcagtgtagatctaaataaagcaatagcatcacaaataaagcatttcattcactgcattctagttgtggtttgtacgcgtgatatcaattcatctatgtcgggtgcggagaaagaggtaatgaaatggcatcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttataactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaagattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtcctgccgcttagcgtgttccgtgtgatagttcaagtacaaccacactcttaagttggtcaaaagaatgctggcttcagttgtaatcaaaactccatcgcatctaatcgttctgaggaaatcatccacggtagcatatgcaaatcccaaccaagcaatgcaactggattgtgtttcaagcaggagaggagagggaagagacggaagaaccatgttaatttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttacagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttacatcgttaattaa

SEQ ID NO: 5 - protein, 235 aa, Artificial Sequence
  [CDS]4349..5053 from SEQ ID NO 4
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 6 - DNA, 37077 bp, Artificial Sequence
  Ad5 including E4 orf4-encoded protein
  LTR 1..103: /note=“L-ITR”
  promoter 321..963: /note=“CMVgal promoter”
  gene 1037..1956: /note=“E1A (delta aa 4-25)”
  misc_feature 1147..1167: /note=“RNAi target site”
  misc_feature 1196..1219: /note=“RNAi target site”
  misc_feature 1994..2074: /note=“heterologous target sites for RNAi inserted into the 3?UTR of E1A, equivalent to E1A-1196..1219, E1A-1147..1167, Prot IX-7409..7429”
  promoter 2084..2220: /note=“promoter for E1B”
  gene 2221..2751: /note=“E1B-19k”
  gene 2526..4016: /note=“E1B-55k”
  misc_signal 3421..4630: /note=“IRES”
  gene 4659..5363: /note=“FLT3L”
  misc_feature 5364..5426: /note=“2A”
  gene 5427..5702: /note=“MIP1A/CCL3”
  misc_feature 5703..5765: /note=“2A”
  gene 5766..6110: /note=“XCL1”
  promoter 6182..6538: /note=“prMinRGC”
  gene 6539..7144: /note=“GAL4-KRAB”
  gene 7388..7810: /note=“Protein IX”
  gene 8976..17898: /note=“Ad-Pol”
  gene 12362..17898: /note=“terminal protein TP”
  gene 17935..19650: /note=“Protein III”
  gene 22621..25479: /note=“hexon”
  gene 26222..27811: /note=“reverse complementary sequence of DNA-binding protein, dbd”
  gene 27840..30263: /note=“100k”
  misc_feature 31645: /note=“delta E3”
  gene 32164..33909: /note=“fiber”
  gene 34036..35199: /note=“reverse complementary sequence for E4 orf6/7”
  gene 35120..35464: /note=“reverse complementary sequence for hybrid E4orf4”
  misc_feature 35475..35826: /note=“inactive E4 orf3 locus”
  misc_feature 35803..35805: /note=“stop codon for E4orf3”
  gene 35838..36231: /note=“reverse complementary sequence for E4 orf2”
  gene 36263..36649: /note=“reverse complementary sequence for E4 orf1”
  LTR 36974..37077: /note=“R-ITR”
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttcccgggtacctataccctctagagtctccgctcggaggacagtactccgctcggaggacagtactccgctcggaggacagtactccgctcggaggacagtactccgctcggaggacagtactccgacctgcaggcatgcaaggtacctataccctctagagtctccgctcggaggacagtactccgctcggaggacagtactccgctcggaggacagtactccgctcggaggacagtactccgctcggaggacagtactccgacctgcaggcatgcaaggtaccatggtgatgcggttttggcagtacatcaatggcggtggatagcggtccgactcacggggatttccaagtctccaccccattgacgtcaatgggagtttgttttggcaccaaaatcaacgggactttccaaaatgtcgtaacaactccgccccacggacgcaaatgggccgtaggcgtgtacggtgggcggtctatataagcccggctggtttagtgaaccgtcagatcctcgactagagtctcgctcggaggacagtactccgctcggaggacagtactccgctcggaggacagtactccgctcggaggacagtactccgctcggaggacagtactccgacctgcaggcatgcaaggttcctcaagaggccactcttgagtgccagcgagtagagttttctcctccgagccgctccgacaccgggactgaaaatgagacatgaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacacacaggaggcggtttcgcagatttttacacacagaagggattgacttactcacttttacacacaggaagcattgtgagctcatatgagacccaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgatcacgcgtttcgaacgatttaaatttaaatatgacagtgctggcgccagcctggagcccaacaacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggctgctgcctggtgcctgcactggcagaggacgcggcggaggacaccccgccctggggagcaggtgccccccgtccccagtccccaggacctgctgcttgtggagcactccggagccacgaacttctctctgttaaagcaagcaggagacgtggaagaaaaccccggtcccatgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctggagctgagtgcctccggagccacgaacttctctctgttaaagcaagcaggagacgtggaagaaaaccccggtcccatgagacttctcatcctggccctccttggcatctgctctctcactgcatacattgtggaaggtgtagggagtgaagtctcagataagaggacctgtgtgagcctcactacccagcgactgccagttagcagaatcaagacctacaccatcacggaaggctccttgagagcagtaatttttattaccaaacgtggcctaaaagtctgtgctgatccacaagccacatgggtgagagacgtggtcaggagcatggacaggaaatccaacaccagaaataacatgatccagaccaagccaacaggaacccagcaatcgaccaatacagctgtgactctgactggctagctcgagaataaaggtaccttattttcattagatttgtgtgttggttttttgtgtggcggcctcgagatgggagagcttgatgccaggcaagtccaggcaggccaggcaagtccaggcaggccaggcaagtccaggcaggccaggcaagtccaggcaggccaggcaagtccaggcaggccaggcaagtccaggcaggccaggcaagtccaggcaggccaggcaagtccaggcaggccaggcaagtccaggcaggccaggcaagtccaggcagggccaggcaagtccaggcaggccaggcaagtccaggcaggccaggcaagtccaggcaggatcgaattcgagctcgtacggtgggagggctatataagcagacctcgtttagtgaaccgtcagatcgcctggagacgaagcttgctagcggatccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgatgactgcagaaatggatatggccctcatgcagggctgtgtgacctttcaagatgtggccatttgcttctcccatgaagaatggagacttcttgatgagactcagaggctcctgtacctcagtgttatgctgcagaactttgcactcataaactcccagggttaggattagtcatatgctgcagtgtagatctaaataaagcaatagcatcacaaataaagcatttcattcactgcattctagttgtggtttgtacgcgtgatatcaattcatctatgtcgggtgcggagaaagaggtaatgaaatggcatcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttataactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaagattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtcctgccgcttagcgtgttccgtgtgatagttcaagtacaaccacactcttaagttggtcaaaagaatgctggcttcagttgtaatcaaaactccatcgcatctaatcgttctgaggaaatcatccacggtagcatatgcaaatcccaaccaagcaatgcaactggattgtgtttcaagcaggagaggagagggaagagacggaagaaccatgttaatttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttacagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttacatcgttaattaa

SEQ ID NO: 7 - DNA, 902 bp, Artificial Sequence
  synTert promoter
  misc_feature 28..258: /note=“insulator of chicken beta-globin”
  protein_bind 259..448: /note=“10x Gal4, ten binding sites for yeast transcription factor Gal4”
  protein_bind 449..511: /note=“2x HRE (hypoxia response elements), two binding sites for hypoxia response factor”
  protein_bind 512..549: /note=“2x E2F, two binding sites for transcription factor E2F”
  protein_bind 550..589: /note=“4x myc, four binding sites for transcription factor c-myc”
  polyA_site 590..600: /note=“polyadenylation sequence”
  promoter 601..801: /note=“minTert, minimal promoter of telomerase subunit of hTert”
  TATA_signal 802..902: /note=“Tata-box of the adenoviral E1A locus”
taactataacggtcctaaggtagcgaaggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaacggaggacagtactccgctcggaggacagtactccgctcggaggacagtactccgctcggaggacagtactccgctcggaggacagtactccgctcggaggacagtactccgctcggaggacagtactccgctcggaggacagtactccgctcggaggacagtactccgctcggaggacagtactccgactcgaccacagtgcatacgtgggctccaacaggtcctcttctgcatacgtgggctccaacacagtcgtggctctttcgcgcaaaaaggatttggcgcgtaaagtcacgtgacgtcacgtgacgtcacgtgacgtcacgtgacacaaataaagcacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagcgtgtagtgtatttatacccggtgagttcctcaagaggccactcttgagtgccagcgagtagagttttctcctccgagccgctccgacaccgggactgaaa

(19) United States
(12) Patent Application Publication
(10) Pub. No.: US 2024/0261350 A1
(43) Pub. Date: Aug. 8, 2024

(54) ADENOVIRUS FOR ANTI-TUMOUR THERAPY

(71) Applicant: MEDIZINISCHE HOCHSCHULE HANNOVER, Hannover (DE)

(72) Inventors: Florian Kühnel, Hannover (DE); Norman Woller, Hannover (DE); Stefan Kubicka, Tübingen (DE)

(21) Appl. No.: 17/927,420

(22) PCT Filed: May 20, 2021
(86) PCT No.: PCT/EP2021/063539

§ 371 Date: Nov. 23, 2022

(30) Foreign Application Priority Data
May 25, 2020 (EP) 20176378.6

(51) Int. Cl. A61K 35/761; A61P 35/00; C12N 15/86

(52) U.S. Cl. CPC A61K 35/761; A61P 35/00; C12N 15/86; C12N 2710/10322; C12N 2710/10332; C12N 2710/10343

(57) ABSTRACT

Adenovirus for use in anti-tumour therapy which has specificity for tumour cells, especially for tumour cells that have reduced activity levels of the tumour suppressor p53. It is known that the majority of tumours characteristically exhibit reduced activity of the tumour repressor p53. In tumour-bearing patients, the adenovirus provides for an effective cross-presentation of tumour antigens, which can be neoantigens. e.g. having low immunogenicity, and hence supports the induction of tumour-specific immune cells, especially of tumour-specific CD8 T− cells. Further, the adenovirus in tumour patients stimulates the non-specific immune response by NK-cells, preferably both locally and systemically, and the adenovirus improves migration of immune cells. e.g. T-cells. NK-cells, and antigen-presenting cells (APC), e.g. dendritic cells, into the tumour, and the adenovirus improves the maturation of immune cells, especially of APC, and improves the cytolysis of tumour cells by immune cells. The adenovirus has an E4 protein which contains an E4 orf4-encoded protein of one of amino acid sequences SEQ ID NO: 1, SEQ ID NO: 2, and SEQ ID NO: 3.

The present invention relates to an adenovirus for use in anti-tumour therapy. The adenovirus has specificity for tumour cells, especially for tumour cells that have reduced activity levels of the tumour suppressor p53. It is known that the majority of tumours characteristically exhibit reduced activity of the tumour repressor p53. In tumour-bearing patients, the adenovirus provides for an effective cross-presentation of tumour antigens, which can be neoantigens, e.g. having low immunogenicity, and hence supports the induction of tumour-specific immune cells, especially of tumour-specific CD8 T-cells. Further, the adenovirus in tumour patients stimulates the non-specific immune response by NK-cells, preferably both locally and systemically, and the adenovirus improves migration of immune cells, e.g. T-cells, NK-cells, and antigen-presenting cells (APC), e.g. dendritic cells, into the tumour, and the adenovirus improves the maturation of immune cells, especially of APC, and improves the cytolysis of tumour cells by immune cells. Further, the adenovirus in a tumour patient provides for a tumour environment which supports the immune response, e.g. by inducing expression of cytokines, e.g. IFN, in the environment of the tumour. The tumour can be a single-cell tumour or a solid tumour.

### STATE OF THE ART

Kühnel et al., Cancer Gene Therapy 28-40 (2004), describe a mutated CMV-promoter (CMVgal) which in the presence of p53 is transcriptionally repressed by a synthetic fusion protein (GAL4-KRAB). The fusion protein GAL4-KRAB which represses the CMVgal-promoter is expressed under the control of a p53-dependent promoter. As a result, the CMV-promoter is active in the absence of p53, making it suitable for use in tumour-specific adenoviral gene therapy.

Gürlevik et al., Nucl Acid Res Vol. 37, No. 12 (2009), describe an adenovirus expressing interfering RNA under the control of a p53-dependent promoter (prMinRGC) to inhibit expression of essential adenoviral genes, e.g. E1A, E1B, E4, pTP, in the presence of p53, e.g. in p53-normal, non-tumour cells. The adenovirus was shown not to replicate in p53-positive cells but to replicate in p53-negative cells representing tumour cells.

Kühnel et al., Molecular Therapy 936-946 (2010), describe a chemotherapy in combination with an adenovirus that is p53-sensitive as it contains the CMVgal promoter which is repressed by a fusion protein GAL4-KRAB, which is expressed under the control of a p53-denpendent promoter (prMinRGC).

### OBJECT OF THE INVENTION

It is an object of the invention to provide an adenovirus that is suitable for use in tumour treatment and which is specific for tumour tissue and induces an effective CD8 T-cell immune response, and which adenovirus preferably also effectively induces an immune response directed against the tumour rather than an immune response directed against the adenovirus. More preferably, the adenovirus is intended to induce an effective immune response against a tumour bearing a tumour antigen that inherently is not highly immunogenic, e.g. a neo-antigen.

### DESCRIPTION OF THE INVENTION

The invention achieves the object by the features of the claims and especially provides adenovirus, especially for use in the treatment of a tumour, the adenovirus having an E4 protein which contains an E4 orf4-encoded protein of one of amino acid sequences
MVLPSLPSPLLLETQSSCIAWLGFAYATVDDFLRTIRCDGVLITTEASILLTNLRVWLY LNYHTEHAKRQDRRRRSVCHARTWFCFRKYDYVRRSIWHDTTTNTISVVSAHSVQ (SEQ ID NO: 1),
MILPSLPSPLLLETQSSCIAWLGFAYATVDDFLRTIKYDEVLITTEASILLTNLRVWLY LNYQTEHTKRQDRRRRSVCHARTWFCFRKYDYVRRSIWHDTTTNTISVVSAHSVQ (SEQ ID NO: 2), and
MILPSLPSPLLLETQSSCIAWLGFAYATVDDFLRTIKYDGVLITTEASILLTNLRVWLY LNYQTEHTKRQDRRRRSVCHARTWFCFRKYDYVRRSIWHDTTTNTISVVSAHSVQ (SEQ ID NO: 3), including an amino acid sequence having a sequence identity or homology of at least 80%, of at least 85%, of at least 90%, of at least 95% to one of, to two of, or to the three of the sequences SEQ ID NO: 1, SEQ ID NO: 2 and SEQ ID NO: 3,
and a DNA encoding an adenovirus containing the coding sequence for an E4 protein containing the E4 orf4 encoded protein.

For the adenovirus for use in the treatment of a tumour, the tumour can express a neo-antigen, which can e.g. have a low antigenicity, because the adenovirus provides for an effective cross-presentation of tumoural antigens to immune cells.

The E4 protein according to the invention comprises or consists of the protein sections encoded by E4 orf1, E4 orf2, E4 orf3, E4 orf4 having one of SEQ ID NO: 1, SEQ ID NO: 2, and SEQ ID NO: 3, E4 orf6 and E4 orf7, wherein E4 orf3 preferably is inactivated, and wherein preferably E4 orf1, E4 orf2, E4 orf3, E4 orf6 and E4 orf7 have the amino acid sequence of the respective wild-type Ad5. A protein section encoded by an inactivated E4 orf3 may be an absent E4 orf3 protein section or a truncated E4 orf3 protein section, e.g. consisting of at maximum N-terminal 10 amino acids, preferably of at maximum N-terminal 7 amino acids or less of the wild-type E4 orf3 encoded protein section.

Further, the invention provides a process for producing the adenovirus of the invention, which process has the advantage of producing the adenovirus at a high titer, because the E4 protein containing one of the E4 orf4 sequences of SEQ ID NO: 1 to NO: 3 does not result in a lower titer, e.g. compared to adenovirus containing the wild-type E4 or compared to wild-type Ad5. Further, it has been found that the adenovirus according to the invention has a high stability e.g. for biotechnological production of the virus.

The E4 encoding sequence preferably contains or consists of the wild-type sequences of Ad5 for orf1, orf2, orf3 or inactivated orf3, an E4 protein containing the orf4 of one of SEQ ID NO: 1, NO: 2 or NO: 3, and wild-type sequences of Ad5 for orf6 and orf7.

It was found that an adenovirus, which preferably is adenovirus serotype 5 (Ad5), comprising in its E4 protein an E4-orf4 encoded protein section of one of SEQ ID NO: 1, SEQ ID NO: 2, and SEQ ID NO: 3, and preferably an inactivated E4 orf3 protein section, has high specificity for cells with reduced activity levels of p53, which is e.g. a reduced or absent expression of p53, e.g. for p53-negative tumour cells, induces an effective immune response, e.g. an effective CD8 T-cell response, which is directed against tumour antigen, and which immune response is less anti-viral e.g. compared to wild-type Ad5, wherein the anti-tumour immune response can also be directed against a tumour neo-antigen that has a low immunogenicity.

In addition, the adenovirus of the invention has been found to be less hepatotoxic, e.g. compared to wild-type Ad5.

Preferably, the virus of the invention in its nucleic acid sequence contains an expression cassette encoding an effector molecule for expression in tumour cells. The expression cassette encoding an effector molecule can e.g. be under the control of an IRES (internal ribosomal entry site) or under the control of a separate promoter, e.g. a promoter that is inactivated by the presence of p53. The effector molecule can comprise or consist of at least one effector molecule, e.g. a fusion protein of two or three effector molecules, which preferably are connected by an intermediate 2A element. Exemplary effector molecules are cytokines, e.g. FLT3L (FMS-like tyrosine kinase 3 ligand), MIP1a (macrophage inflammatory protein 1a), XCL1 (chemokine (C Motif) ligand).

Generally preferred, the adenovirus of the invention contains a double-stranded nucleic acid molecule in which the arrangement of the genetic elements corresponds to their arrangement in the wild-type Ad5, more preferably with an expression cassette for an effector molecule arranged directly adjacent to the 3′ terminus of coding region for E1B, and directly adjacent to the 3′ terminus of the expression cassette for an effector molecule an expression cassette for inhibitory RNA directed against the E1A encoding DNA sequence, against the E1B encoding DNA sequence, against the E1B promoter, against the TP encoding DNA sequence, against the Pol encoding sequence, and/or against the E4 encoding DNA sequence.

Preferably, the adenovirus encoding DNA from 5′ to 3′ comprises or consists of a left-end inverted terminal repeat (L-ITR), a first promoter, a coding sequence for E1A, an E1B promoter controlling a coding sequence for E1B, an expression cassette for at least one effector molecule, preferably an expression cassette encoding inhibitory RNA (RNAi) that are specific for RNAi target sites within the E1A encoding sequence and/or within the E1B promoter and/or within the E1B encoding sequence, vector backbone sequence, and on the opposite DNA strand an expression cassette for E4 comprising the orf4 of one of SEQ ID NO: 1, NO: 2 and NO: 3, the expression cassette for E4 containing in functional relationship a promoter driving expression of E4, and a right-end inverted terminal repeat (R-ITR). The arrangement of the L-ITR, E1A, a promoter controlling expression of E1B, preferably of an expression cassette for at least one effector molecule on one DNA strand, and preferably of coding sequences for inhibitory RNA under the control of a promoter which is activated by the presence of p53, e.g. the prMinRGC promoter, on one DNA strand, and arrangement of the expression cassette for E4 on the opposite DNA strand corresponds to the arrangement of the R-ITR, E4-promoter, and of the orfs constituting the coding sequence for E4 from 5′ to 3′ on the opposite strand. The expression cassette for an E4-orf4 encoded protein is arranged on the DNA strand opposite to the strand encoding the L-ITR, the first promoter, the E1A encoding sequence and the E1B promoter and the E1B encoding sequence. The L-ITR is arranged at one terminus of the DNA and the R-ITR is arranged at the terminus of the DNA opposite of the L-ITR.

The first promoter preferably is CMVs, which has the advantage of becoming activated following infection of a cell with a time delay that allows for the effective inhibition of the E1A encoding sequence, the E1B promoter and of the E1B encoding sequence by inhibitory RNA molecules that are expressed under the control of prMinRGC.

Preferably, the coding sequence for E1A, the E1B promoter, and the coding sequence for E1B contain RNAi target sites for binding inhibitory RNA, e.g. shRNA, and the adenovirus encoding DNA contains an expression cassette for inhibitory RNA, e.g. for shRNA, which bind to the RNAi target sites, which expression cassette preferably is under the control of a promoter which is activated by presence of p53, e.g. the promoter prMinRGC.

The coding sequences for inhibitory RNA, which inhibit expression of the adenoviral genes, e.g. of E1A, E1B, E4, pTP, Pol, are preferably expressed under the control of the prMinRGC promoter. The prMinRGC promoter is preferred as the promoter which is activated by the presence of p53. The inhibitory RNA and their target sequences preferably are those described by Gürlevik et al. (2009), quoted above.

In the alternative or in addition to the combination of RNAi arranged under the control of the prMinRGC promoter and CMVs as the first promoter, the first promoter can be a CMV promoter (CMVgal) which in the presence of p53 is transcriptionally repressed by a synthetic fusion protein (GAL4-KRAB), and the DNA encoding the adenovirus comprising an expression cassette encoding the fusion protein GAL4-KRAB under the control of a promoter that is activated by presence of p53. This embodiment also provides for a tight control of expression of E1A and E1B such that these proteins are effectively repressed in the presence of p53 in an infected cell, but that these proteins are effectively expressed in a target cell having reduced activity levels of p53. The CMVgal promoter and the GAL4-KRAB preferably are those described by Kühnel et al., Cancer Gene Therapy 28-40 (2004), quoted above.

The coding sequence for E1A preferably encodes E1A of Ad5, in which amino acids No. 4 to No. 25 of the wild-type sequence are deleted, e.g. amino acids No. 727-1646 of SEQ ID NO: 4. In the viral DNA of SEQ ID NO: 4, the E4 orf4 encoded protein of the invention is contained in reverse complementary orientation, as it is usual for the Ad5. The E4 region is read from the promoter adjacent the left ITR, i.e. from right to left, also reversing the way of numbering E4 genes. The E4 orf4 encoding DNA section contains splice sites, and therefore the reverse complementary sequence does not directly present codons.

The vector backbone sequence comprises or consists of the following elements, e.g. from 5′ to 3′ DNA-binding protein (dbp), terminal protein (TP), polymerase (Pol), Penton (Pent), Hexon (Hex), and Fiber (Fiber).

Due to the specificity of the adenovirus for tumour cells, which are preferably characterized by lower content of p53 than average normal cells, e.g. non-tumour cells, or absence of p53, or presence of dysfunctional p53, the adenovirus is suitable for use in the treatment of a tumour, preferably of a tumour that has a lower presence or lower expression of p53 or an absent presence or absent expression of p53, or presence of dysfunctional p53 e.g. in comparison to non-tumour cells. The specificity of the adenovirus for tumour cells, which especially are p53-deficient cells, in combination with the inhibition of the expression of E1A, E1B in the presence of p53, and the adenovirus preferably containing an expression cassette for at least one effector molecule in cells that express p53 at normal levels makes the adenovirus of the invention suitable for use in the treatment of a tumour.

The adenovirus of the invention is also for use in the treatment of a tumour in combination with anti-PD1/PD-L1 immune therapy. Anti-PD1/PD-L1 immune therapy can be by e.g. Nivolumab, Pembrolizumab, Atezolizumab or a combination of at least two of these.

In the Figures

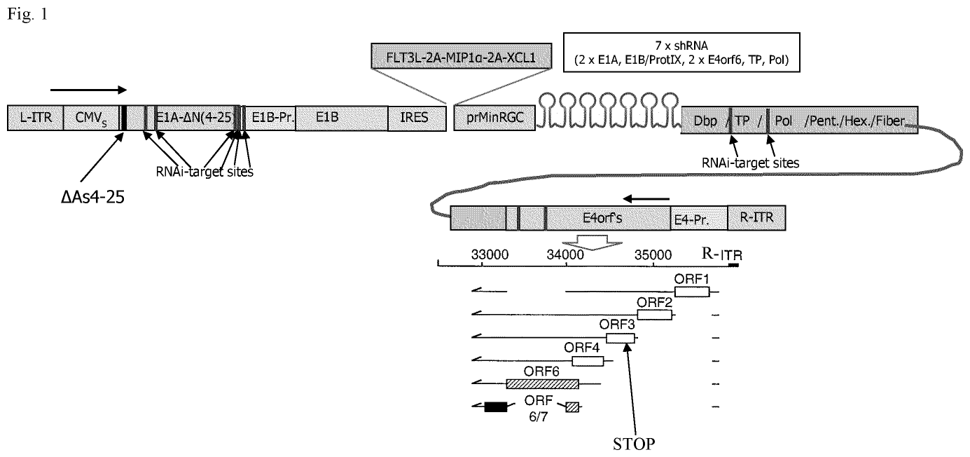

FIG. 1 schematically shows the elements of a preferred embodiment of the adenovirus of the invention,

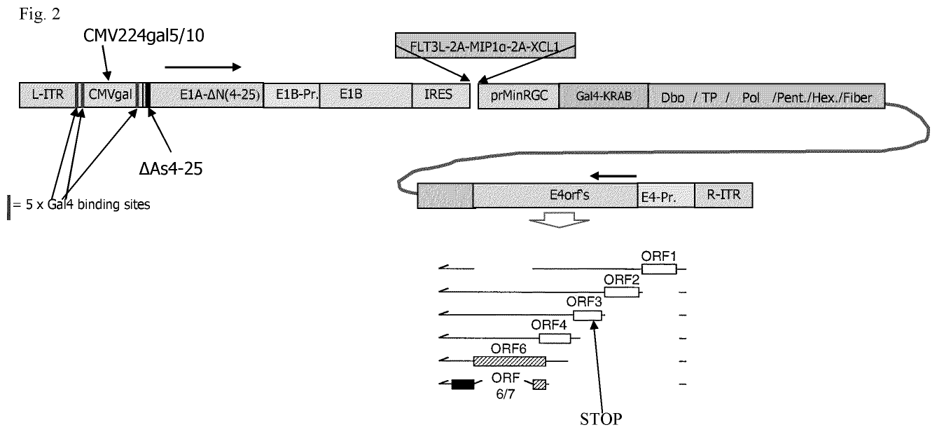

FIG. 2 schematically shows the elements of a further embodiment of the adenovirus of the invention,

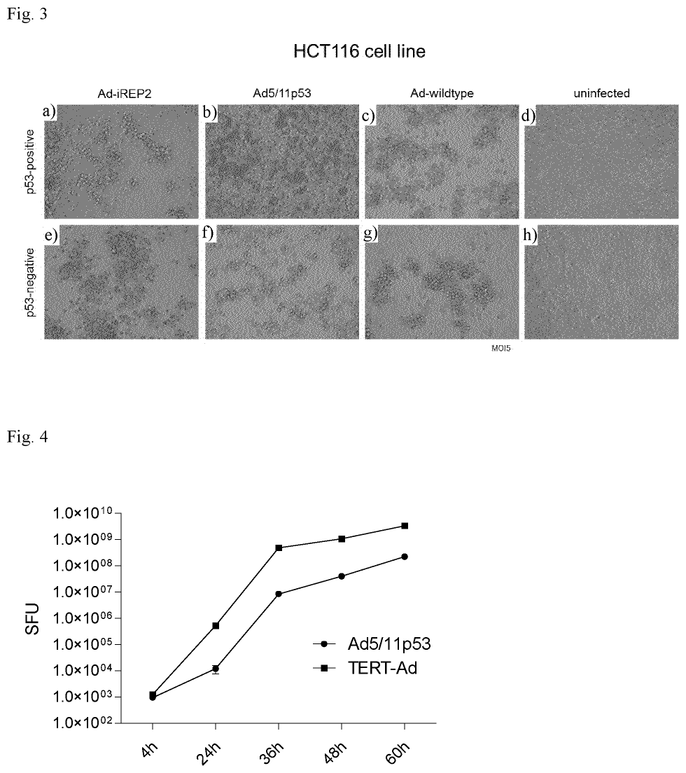

FIG. 3 a) to h) shows light-microscopic pictures of cultivated cells after infection with adenovirus,

FIG. 4 shows the replication kinetics of an adenovirus of the invention and of a comparative adenovirus,

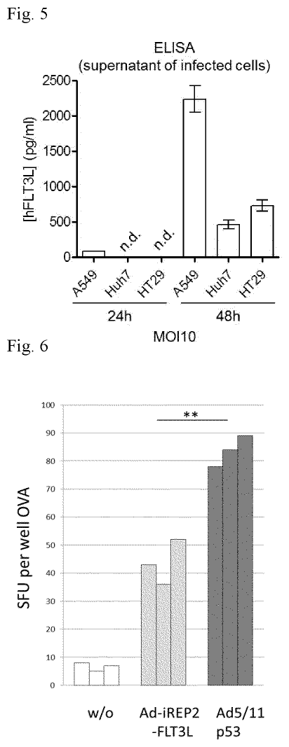

FIG. 5 shows ELISA results for expression of an effector molecule from different cells infected with an embodiment of the adenovirus of the invention,

FIG. 6 shows the results of an ELISPOT assay after tumour treatment using an adenovirus of the invention,

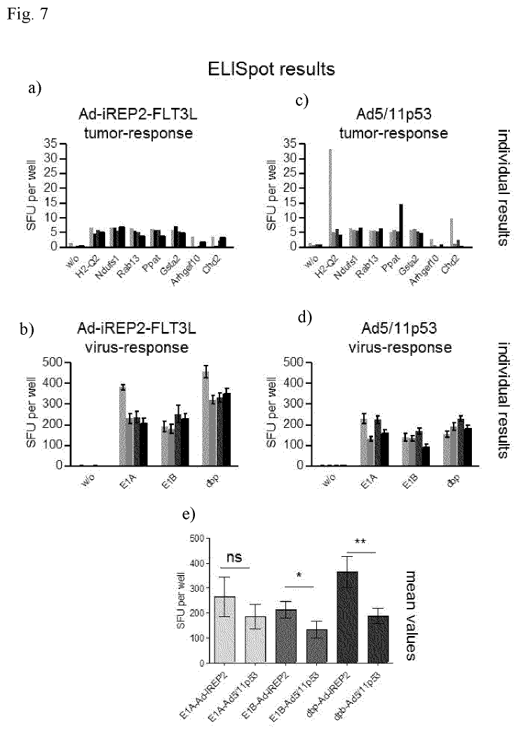

FIG. 7 a) to e) shows results of an analysis of the immune response against tumour bearing neo-antigens after use of an adenovirus of the invention, and

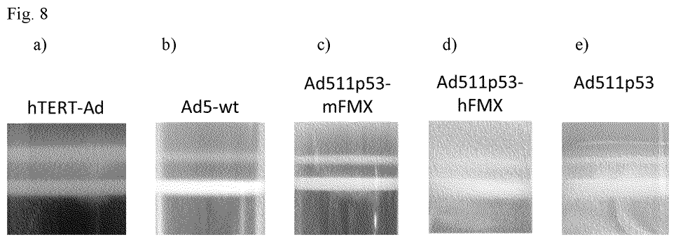

FIG. 8 a) to e) show the results of CsCl gradient separation of defect and intact adenovirus.

### Example: Generating an Adenovirus Having Higher Specificity for p53-Deficient Cells

As an example of tumour cells, the p53-deficient H1299 lung carcinoma cells were used as host cells, which were co-infected with adenovirus Ad5, adenovirus Ad4, and co-infected with adenovirus Ad11, or other alternative serotypes of adenovirus, in the presence of the mutagen ethylmethyl sulfonate. As an alternative, the p53-deficient H1299 lung carcinoma cells were co-infected with adenovirus Ad3, adenovirus Ad5, and optionally additionally co-infected with Ad11 and Ad 21, in the presence of the mutagen ethylmethyl sulfonate. The adenovirus Ad5 contained the expression cassette for inhibitory RNA as described by Gürlevik et al. (2009) under the control of the p53-dependent promoter prMinRGC, rendering the expression of E1A and E1B dependent on the presence of p53, and hence rendering the Ad5 dependent on p53, respectively dependent on healthy p53 cellular levels. Following complete lysis of the host cells by viruses, the viruses were passaged to newly seeded host cells. This was repeated for 12 cycles of passaging after complete lysis. The resulting virus samples were diluted to limitation for isolating single clones, which were purified. For identification of possible genetic alterations of the viral clones, two viral DNA sections were amplified by PCR and the amplificates were tested by digestion with restriction enzymes cutting in wild-type Ad4 and in wild-type Ad11, but not in the Ad5. This restriction fragment analysis identified virus clones that were subsequently sequenced and showed a coding sequence for an E4 orf4 of SEQ ID NO: 1, of SEQ ID NO: 2, and of SEQ ID NO: 3, respectively. In addition, it was found in some clones that the E4 orf3 was inactivated by an insertion mutation generating a stop codon followed by a frameshift, truncating the transcript of E4 orf3 to 7 amino acids.

Further analysis revealed that these E4 proteins containing an E4 orf4 of SEQ ID NO: 1, of SEQ ID NO: 2, or of SEQ ID NO: 3 are hybrid E4 orf4 protein sections of an N-terminal portion from the E4 orf4 of Ad11 with a C-terminal portion from the E4 orf4 from Ad5 (SEQ ID NO: 1), or of an N-terminal portion from the E4 orf4 of Ad3 with a C-terminal portion from the E4 orf4 from Ad5 (SEQ ID NO: 2), or of an N-terminal portion from the E4 orf4 of Ad21 with a C-terminal portion from the E4 orf4 from Ad5 (SEQ ID NO: 3).

In order to generate a preferable adenovirus suitable for the treatment of a tumour, a schematic of which is shown in FIG. 1, the Ad5 containing one of the E4 orf4 in its E4 encoding gene was additionally provided with an expression cassette encoding at least one effector molecule, exemplified in FIG. 1 by a coding sequence for a fusion protein FLT3LK-2A-MIP1a-2A-XCL1 in which the single effector molecules are separable by the intermediate 2A element, or exemplified by the coding sequence for human FLT3L (hFLT3L) under the control of an IRES sequence, each optionally followed by a poly adenylation signal (pA). The expression cassette for the at least one effector molecule was arranged directly adjacent to the 3′-terminus of the E1B encoding sequence. The expression cassette for inhibitory RNA (sh-RNA) under the control of the p53-dependent promoter prMinRGC (minRGC) was arranged in 3′ directly adjacent the expression cassette encoding at least one effector molecule.

Further, in the Ad5, the wild-type promoter of the E1A was replaced by a shortened CMV promoter (CMVs having 245 bp). The CMVs promoter starts transcription of the viral elements that are arranged under its control, which is seen as an advantage especially in combination with the expression cassette for inhibitory RNA directed against the viral elements which are arranged under the control of the CMVs promoter. The viral elements arranged under the control of the CMVs promoter from 5′ to 3′ are the coding sequence for E1A, the E1B promoter and the coding sequence for E1B (E1A-ΔN(4-25)-E1B-Pr.-E1B), and preferably the expression cassette for the at least one effector molecule. Therein, the E1A encoding sequence as preferred is devoid of the N-terminal amino acids No. 4 to No. 25 (ΔN(4-25)) of the wild-type E1A.

The target sequences of the inhibitory RNA molecules are E1A, E1B, dbp, TP and Pol, the target sites (RNAi-target sites) are indicated.

As a further alternative, for expression control of E1A, the wild-type promoter of the E1A was replaced by a synthetic promoter having SEQ ID NO: 7, which is termed synTert promoter.

The viral elements which in FIG. 1 are designated as Backbone preferably have their wild-type arrangement and their wild-type sequence of Ad5 and are arranged between the expression cassette encoding the inhibitory RNA molecules and the E4 encoding sequence.

These elements are arranged between a left-end inverted terminal repeat (L-ITR) and a right-hand inverted terminal repeat (R-ITR).

In FIG. 1 and FIG. 2, the orfs of E4 are schematically depicted in one DNA molecule comprising the adenovirus, and, in accordance with the arrangement on the opposite strand, the orfs of E4 are transcribed from the right to left, as indicated by the arrows.

In the coding sequence for E4, the orf4 encodes one of SEQ ID NO: 1, SEQ ID NO: 2, and SEQ ID NO: 3, and preferably, the orf3 is inactivated, e.g. by a stop codon (STOP) generated close to the N-terminus of orf3, resulting in e.g. an orf3 encoding at maximum the N-terminal 8 or less amino acids of orf3. In FIG. 1, the E4 promoter (E4-Pr.) the coding sequences for E4 are shown from left to right as they are arranged together with the R-ITR on the complementary DNA strand. The numbers indicate the length in bp.

SEQ ID NO: 4 gives the DNA sequence of this exemplary Ad5, with the nucleotide sections for:

| element | Nt sections (No . . . No) |
|---|---|
| L-ITR | 1 . . . 103 |
| R-ITR | 36919 . . . 37021 |
| CMVs | 380 . . . 625 |
| ElA gene (ΔAs4-25-variant) | 727 . . . 1646 |
| RNAi target site | 837 . . . 857 |
| RNAi target site | 886 . . . 909 |
| heterologous target sites for RNAi inserted into | 1684 . . . 1764 |
| the 3′UTR of E1A, equivalent to E1A 886 . . . 909, | |
| E1A 837 . . . 857, Prot IX 7353 . . . 7374 | |
| Promoter for E1B | 1813 . . . 1910 |
| E1B-19k | 1911 . . . 2441 |
| E1B-55k | 2216 . . . 3706 |
| IRES | 3731 . . . 4320 |
| FLT3L (cds) | 4349 . . . 5053 |
| 2A | 5054 . . . 5116 |
| MIP1A/CCL3 | 5117 . . . 5392 |
| 2A | 5393 . . . 5455 |
| XCL1 | 5456 . . . 5800 |
| prMinRGC | 5872 . . . 6230 |
| shRNA network (complete) | 6259 . . . 7064 |
| Inhibitory shRNA for E1A (1) | 6259 . . . 6321 |
| Inhibitory shRNA for E1A (2) | 6387 . . . 6444 |
| Inhibitory shRNA for Prot IX | 6511 . . . 6567 |
| Inhibitory shRNA for E4orf6 (1) | 6634 . . . 6690 |
| Inhibitory shRNA for E4orf6 (2) | 6756 . . . 6816 |
| Inhibitory shRNA for TP | 6882 . . . 6939 |
| Inhibitory shRNA for AdPol | 7006 . . . 7064 |
| Protein IX, part of backbone | 7332 . . . 7754 |
| Polymerase; Ad-Pol, part of backbone | 8920 . . . 17842 |
| Terminal Protein; TP, part of backbone | 12306 . . . 17842 |
| Prot III: Penton, part of backbone | 17878 . . . 19593 |
| hexon, part of backbone | 22563 . . . 25421 |
| DNA-binding protein, dbd, part of backbone | 26164 . . . 27753 |
| 100k, part of backbone | 27782 . . . 30205 |
| ΔE3 (2681 bp) | at 31589 (equivalent to del. of 28137 . . . |
| | 30818 in the wildtype virus) |
| fiber, part of backbone | 32108 . . . 33853 |
| E4orf6/7 | 33980 . . . 35143 |
| hybrid E4orf4, Ad5/Ad11; Seq ID NO: 1 | 35064 . . . 35408 |
| Stop codon E4orf3 | 35747 . . . 35749 |
| Inactive E4orf3 locus | 35419 . . . 35770 |
| E4orf2 | 35782 . . . 36175 |
| E4orf1 | 36207 . . . 36593 |
| r-ITR | 36919 . . . 37021 |
| Total length 37021 bp | |
| Target sequences of the shRNAs in the network | 5′ to 3′: 886, 837, 7354, 34123, |
| | 34609, 13375, 10085 |

Further deviations from the sequence of Ad5, accession No. AC_000008, are indicated in the attached sequence protocol.

As an alternative to the inhibitory shRNA sections for repressing the virus in the presence of p53, i.e. in healthy cells, using a p53-dependent promoter (prMinRGC) for expressing inhibitory shRNAs against viral genes as shown in FIG. 1, a p53-dependent promoter can be used to control essential viral genes for expression only in absence of p53 in infected cells. An embodiment is shown in FIG. 2, wherein a constitutive promoter, e.g. the CMVgal promoter is arranged for expression of essential viral genes, which CMVgal promoter is repressed by a fusion protein, e.g. GAL4-KRAB, which is expressed under the control of a p53-dependent promoter, e.g. prMinRGC. In SEQ ID NO: 6, the elements of this embodiment are shown.

| element | Nt sections (No . . . No) |
|---|---|
| L-ITR | 1 . . . 103 |
| CMVgal promoter (CMV224gal10/5) | 321 . . . 963 |
| E1A gene (ΔAs4-25 variant) | 1037 . . . 1956 |
| RNAi target site | (1147 . . . 1167) |
| RNAi target site | (1196 . . . 1219) |
| heterologous target sites for RNAi inserted into | (1994 . . . 2074) |
| the 3′UTR of E1A, equivalent to E1A | |
| 1196 . . . 1219, E1A 1147 . . . 1167, Prot IX | |
| 7409 . . . 7429 | |
| Promoter for E1B | 2084 . . . 2220 |
| E1B-19k | 2221 . . . 2751 |
| E1B-55k | 2526 . . . 4016 |
| IRES | 3421 . . . 4630 |
| FLT3L | 4659 . . . 5363 |
| 2A | 5364 . . . 5426 |
| MIP1A/CCL3 | 5427 . . . 5702 |
| 2A | 5703 . . . 5765 |
| XCL1 | 5766 . . . 6110 |
| prMinRGC | 6182 . . . 6538 |
| GAL4-KRAB | 6539 . . . 7144 |
| Protein IX (part of backbone) | 7388 . . . 7810 |
| Polymerase (Ad-Pol, part of backbone) | 8976 . . . 17898 |
| Terminales Protein (TP, part of backbone) | 12362 . . . 17898 |
| Prot III: Penton, part of backbone | 17935 . . . 19650 |
| Hexon, part of backbone | 22621 . . . 25479 |
| DNA-binding protein, dbd, part of backbone | 26222 . . . 27811 |
| 100k, part of backbone | 27840 . . . 30263 |
| ΔE3 (2681 bp) | at 31645 (equivalent to del. of 28137 . . . |
| | 30818 in the wildtype virus) |
| Fiber, part of backbone | 32164 . . . 33909 |
| E4orf6/7 | 34036 . . . 35199 |
| hybrid E4orf4, Ad5/Ad11; Seq ID NO: 1 | 35120 . . . 35464 |
| Stop codon E4orf3 | 35803 . . . 35805 |
| Inact. E4orf3 locus | 35475 . . . 35826 |
| E4orf2 | 35838 . . . 36231 |
| E4orf1 | 36263 . . . 36649 |
| R-ITR | 36974 . . . 37077 |
| total length 37077 bp | |

The embodiment of the viral vector of SEQ ID NO: 4 (Ad5/11p53) was used to infect the p53-positive host cell-line HCT116 and a p53-deficient but otherwise isogenic host cell-line representing a tumour cell, each with a multiplicity of infection (MOI) of 5. As a comparison, the wild-type Ad5 (Ad-wildtype) and an adenovirus having the complete wild-type E4 (Ad-iREP2) were used for infection. FIG. 3 a) to d) show microscopic pictures of the p53-positive host cell-line, and FIG. 3 e) to h) show the p53-negative host cell-line. In these pictures, non-adherent cells are cytopathic and indicate lysis. FIGS. 3d) and 3 h) show the host cell-line without infection. It was found that the adenovirus of the invention in p53-negative host cells (FIG. 3 f) shows a significantly higher lysis than in p53-positive host cells (FIG. 3 b), indicating its high selectivity for p53-negative cells. In comparison to comparative Ad-iREP2 and wild-type Ad5, the adenovirus of the invention showed an oncolytic effect on p53-negative cells that is comparable or higher, and which has higher specificity for p53-negative cells.

As an example of the adenovirus according to the invention, the adenovirus containing the DNA of SEQ ID NO: 4 was produced e.g. in cultivated Huh7 cells, which are p53-deficient and contain a mutation (p53del+p53Cys220), and as a comparison, adenovirus containing (hTert-Ad), and as a further comparison, wild-type Ad5. As shown in FIG. 4, the adenovirus according to the invention (Ad5/11p53) having SEQ ID NO: 4 in these cells had a replication kinetic that is retarded 100-fold in the first 24 h compared to hTert-Ad. The replication of the adenovirus according to the invention (Ad5/11p53) was retarded ca. 1000-fold in the first 24 h compared to wild-type Ad5. The retarded replication kinetics of the adenovirus according to the invention are presently considered to provide for an enhanced safety of the medical administration of the adenovirus, e.g. because the lower initial viral replication rate can result in less side-effects.

As an example of adenovirus according to the invention, an embodiment was used which corresponds to SEQ ID NO: 4 except for the effector molecule being human FLT3L (hFLT3L) only under the control of an IRES and with a poly-adenylation element at its 3′-end. As host cells, A549, Huh7 and HT29 cells, all p53-negative, were infected with a MOI of 10. The supernatant of these cell cultures was analysed by an ELISA for hFLT3L. The results, depicted in FIG. 5, show that after 48 h post infection, the effector hFLT3L is expressed from these exemplary cells.

The immunologic properties of the adenovirus of the invention was tested by infecting CMT64-Ova tumours (p53-negative) which express OVA as a model tumour antigen. The CMT64-Ova tumours were grown subcutaneously in C57BL/6 mice, and 1×10⁹ infectious particles of the adenovirus were injected intra-tumourally. 48 h post infection, mice were killed and splenocytes were isolated and antigen-specific (anti-OVA) T-cell responses were measured in an ELISpot assay. In the ELISpot assay, IFN-gamma antibodies were immobilised to the plate, splenocytes from C57BL/6 mice were added to the plates and stimulated with the OVA peptide SIINFEKL. After at least 24 hours of stimulation, splenocytes were removed and IFN-gamma spots were measured by HRP immunostaining.

FIG. 6 shows the results of an ELISPOT assay for 3 individual mice bearing the CMT64-Ova tumour, without infectious particles (w/o), for the comparative adenovirus having the complete wild-type E4 expressing FLT3L as the effector molecule (Ad-iREP2-FLT3L), and an embodiment of the invention (Ad5/11 p53) also expressing FLT3L and which differs from the comparative adenovirus only in the E4 orf4 encoding SEQ ID NO: 1 and E4 orf3 being inactive. The results show that the adenovirus of the invention induces a significantly higher antigen-specific CD8 T-cell response than the comparative adenovirus. This shows that the E4 orf4 of the invention effectively increases the T-cell response specifically directed against the tumour-antigen.

In a further test, a syngeneic lung tumour was used as a subcutaneous model tumour in mice. This lung tumour expresses neo-antigens, which are significantly less immunogenic than the strong immunogenic OVA antigen. These tumour-specific neoantigens were mutated forms of the genes H2-Q2, Ndufs1, Rab13, Ppat, Gsta2, Arhgef10, and Chd2 (H2-Q2-D244E, Ndufs1-V491A, Rab13-K196N, Ppat-1208M, Gsta2-Y9H, Arhgef10-M207I, and Chd2-T647A.

The analysis of the antiviral immune responses against E1A, E1B and against the adenoviral DNA-binding protein (dbp) from these mice could show that the adenovirus of the invention induced a lower immune response against viral E1B and dbp, and to a lesser extent against E1A. It was found that only the adenovirus of the invention induced significant T-cell responses directed against the tumour neo-antigens, whereas the comparative adenovirus did not induce tumour-antigen specific T-cell responses but induced stronger anti-viral immune responses.

The results are shown in FIG. 7 a) and b) for the comparative adenovirus Ad-iREP2-FLT3L and in FIG. 7 c) and d) for the adenovirus according to the invention (Ad5/11p53), and FIG. 7 e) shows mean values.

The adenovirus of the invention also has increased stability during replication, which contributes to induction of a lower anti-viral immune response and of a higher anti-tumoral immune response. The stability of the adenovirus of the invention was tested by determining the proportion of intact viral particles to defect viral particles. The expression cassette encoding the effector molecule was provided with the coding sequence for hFLT3L only, for the combination of hFLT3L-2A-CCL3-2A-XCL1, wherein also XCL1 and CCL3 had the human sequence, and for the combination of mFLT3L-2A-CCL3-2A-XCL1, wherein also XCL1 and CCL3 had the murine sequence. The stability was determined by isolating the virus from cell culture producing the adenovirus, by freeze-thaw cycles followed by CsCl ultra-centrifugation and determining the thickness of the band of intact virus (lower light band) in relation to the thickness of the band of defect virus (upper light band) as shown in FIG. 8 a) for the comparative adenovirus hTERT, b) for wild-type Ad5 (Ad5-wt), c) for an adenovirus of the invention having SEQ ID NO: 4 and encoding murine FMX (mFMX) as the effector molecule, d) for an adenovirus of the invention having SEQ ID NO: 4 or SEQ ID NO: 6 and encoding human FMX (hFMX), and in e) for an adenovirus of the invention having SEQ ID NO: 4 or SEQ ID NO: 6 and encoding only hFLT3L (Ad5/11p53) as the effector molecule. These embodiments, comprising a coding sequence for one effector molecule or a coding sequence for three effector molecules, showed a stability similar to or higher than the stability of wild-type Ad5.

1. Adenovirus comprising an E4 protein comprising an E4 orf4 having an amino acid sequence of at least 80% homology to SEQ ID NO: 1, to SEQ ID NO: 2, and/or to SEQ ID NO: 3.

2. Adenovirus according to claim 1, characterized in that the E4 protein consists of E4 orf1, E4 orf2, inactivated E4 orf3, E4 orf4 having one of SEQ ID NO: 1, SEQ ID NO: 2, and SEQ ID NO: 3, E4 orf6 and E4 orf7.

3. Adenovirus according to claim 1, comprising a nucleic acid coding sequence comprising a terminally arranged left-end ITR (L-ITR), a first promoter functionally arranged at a nucleic acid encoding E1A, an expression cassette of an E1B promoter functionally arranged at the E1B encoding sequence, an expression cassette encoding an effector molecule, an E4 promoter functionally arranged at the nucleic acid sequence encoding the E4, an expression cassette encoding the E4 protein under the control of an E4 promoter, and a terminally arranged right-end ITR (L-ITR) opposite the left-end ITR (L-ITR).

4. Adenovirus according to claim 1, comprising at least one expression cassette for inhibitory RNA encoding inhibitory RNA directed against the E1A encoding DNA sequence, against the E1B encoding DNA sequence, against the E1B promoter, against the TP encoding DNA sequence, against the Pol encoding sequence, and/or against the E4 encoding DNA sequence, which inhibitory RNA are arranged under the control of a promoter that is activated by the presence of p53.

5. Adenovirus according to claim 1, comprising at least one expression cassette for viral genes comprising DNA-binding protein, terminal protein, polymerase, Penton, Hexon and Fiber, under the control of a constitutive promoter, which is repressable by a fusion protein which is expressed under the control of a p53-dependent promoter.

6. Adenovirus according to claim 3, wherein the first promoter is a CMVs promoter having a sequence of nucleotides No. 380 . . . 625 of SEQ ID NO: 4.

7. Adenovirus according to claim 3, wherein the first promoter is a CMVgal promoter having a sequence of nucleotides No. 321 . . . 963 of SEQ ID NO: 6, and wherein the DNA comprises an expression cassette encoding GAL4-KRAB having a sequence of nucleotides No. 6539 . . . 7144 of SEQ ID NO: 6 under the control of a promoter that is activated by the presence of p53.

8. Adenovirus according to claim 3, wherein the first promoter is a promoter having SEQ ID NO: 7.

9. Adenovirus according to claim 3, wherein the E1A encoding DNA sequence is devoid of the coding DNA sequence encoding amino acids No. 4 to 25 of the wild-type E1A amino acid sequence.

10. Adenovirus according to claim 3, wherein the promoter that is activated by the presence of p53 is the prMinRGC promoter having a nucleotide sequence of No. 5872 to 6230 of SEQ ID NO: 4.

11. Adenovirus according to claim 3, wherein the expression cassette encoding an effector molecule encodes at least one cytokine.

12. Adenovirus according to claim 3, wherein the expression cassette encoding an effector molecule encodes a fusion protein comprising at least two of or the three of FLT3L and MIP1a and XCL1, and a 2A element between each two of these.

13. Adenovirus according to claim 1 for use in the treatment of a tumor that has p53 at a reduced activity level.

14. Adenovirus for use in the treatment of a tumour that has p53 at a reduced activity level according to claim 13, for inducing a tumour-specific T-cell immune response against the tumor.

15. Adenovirus according for use in the treatment of a tumor according to claim 13, the use being intra-tumoral administration.

16. Process for producing adenovirus in cultivated mammalian cells, the adenovirus being one according to claim 1.
